(19) United States
(12) Patent Application Publication
(10) Pub. No.: US 2011/0117058 A1
(43) Pub. Date: May 19, 2011

(54) METHOD OF TREATING GENETIC DISORDERS

(75) Inventor: Alberto Auricchio, Napoli (IT)

(73) Assignee: FONDAZIONE TELETHON, Roma (IT)

(21) Appl. No.: 12/935,806

(22) PCT Filed: Mar. 30, 2009
(86) PCT No.: PCT/EP2009/002293

§ 371 Date: Oct. 21, 2010

Related U.S. Application Data
(60) Provisional application No. 61/041,746, filed on Apr. 2, 2008.

(51) Int. Cl. A61K 48/00; C12N 15/63; A61P 27/02

(52) U.S. Cl. 424/93.2; 435/320.1

(57) ABSTRACT

A method for the treatment of diseases associated with mutations in MYO7A or CEP290 genes, especially the Usher Syndrome type IB and Leber congenital amaurosis, by administering to a subject in need thereof an adeno-associated viral vector encoding a MYO7A or a CEP290 protein; genetic constructs and adeno-associated viral vectors for use in this method.

The present invention provides methods and compositions for the treatment of sensorineural diseases associated with mutations in MYO7A and CEP290 genes, in particular the Usher Syndrome type IB (USH) and Leber congenital amaurosis (LCA), by administering to a subject in need thereof an adeno-associated viral vector encoding the MYO7A or CEP290 proteins in the target retinal cells. The invention also includes genetic constructs and adeno-associated viral vectors for use in this method.

### BACKGROUND OF THE INVENTION

Originally described by Leber in 1869, Leber congenital amaurosis (LCA) is an autosomal recessive disease distinct from other retinal dystrophies and responsible for congenital blindness. Leber congenital amaurosis (LCA) (MIM 204000) is characterized by severe or complete loss of visual function apparent early in infancy with failure to follow visual stimuli, nystagmus, and roving eye movements. Affected individuals have an extinguished electroretinogram and eventually develop abnormalities of the ocular fundus including a pigmentary retinopathy. LCA is inherited as an autosomal recessive trait. Aside from helping the patient adapt to life with no visual function, there is no treatment.

Retinitis pigmentosa is the name given to a set of heritable degenerations of the retina. The Usher syndrome is one of the several forms of retinitis pigmentosa and is characterized by retinal degeneration and hearing loss. Usher syndrome has been divided into three major types according to clinical findings. In Usher syndrome type I, retinitis pigmentosa is associated with vestibular ataxia and profound congenital deafness; in Usher syndrome type II, there is retinitis pigmentosa with partial hearing loss; and in Usher syndrome type III, there is retinitis pigmentosa and progressive hearing loss. Most cases of Usher syndrome type II are due to a gene on chromosome 1q41. Most cases of Usher syndrome type III are due to an unidentified gene on chromosome 3q21-24. At least six genes (on chromosomes 10q, 11p15.1, 11q13.5, 14q32, 21q21, and elsewhere) can cause Usher syndrome type I, one of which encodes myosin VIIa. The three types of Usher syndrome together have a combined prevalence of around 5 per 100,000, which corresponds to about 5 to 15 percent of all cases of retinitis pigmentosa.

### DESCRIPTION OF THE INVENTION

The invention is based on the finding that the administration, preferably intraocularly, of MYO7A- or CEP290-encoding adeno-associated viral vectors with AAV5 capsids results in the expression of functional proteins in the targets cells/tissues (photoreceptors for CEP290 and retinal pigment epithelium plus photoreceptors for MYO7A) and in significant and stable morphological and functional improvement of the affected retinas. In particular it has been found that subretinal delivery of rAAV2/5-MYO7A or rAAV215-CEP290 in animal models of Usher IB (shaker I mice, Gibson, F. et al., S.D. 1995. “A type VII myosin encoded by the mouse deafness gene shaker-1”, Nature 374:62-64)]) or LCA due to CEP290 mutations (rd12 mice, Chang B, et al. “In-frame deletion in a novel centrosomal/ciliary protein CEP290/NPHP6 perturbs its interaction with RPGR and results in early-onset retinal degeneration in the rd16 mouse”, Hum Mol Genet. 2006 Jun. 1; 15(11):1847-57. Epub 2006 Apr. 21) results in significant correction of photoreceptor and RPE morphology (photoreceptor cell count, Rhodopsin localization in photoreceptors, RPE melanosomes) and function (retinal electrical activity as measured by electroretinograms).

These findings provide a valuable therapeutic approach to Usher Syndrome type IB (USH) and Leber congenital amaurosis (LCA).

Accordingly, in a first aspect the invention is directed to a method for correcting retinal abnormalities and/or retinal function in a mammalian subject, particularly in a human individual affected by a disease associated with mutations in MYO7A or CEP290 genes, said disease being preferably selected from Usher Syndrome type IB and Leber congenital amaurosis, the method of the invention comprising the steps of:

1) providing a recombinant adeno-associated viral (AAV) vector with AAV5 capsid, said vector carrying an expression cassette which contains a nucleic acid molecule encoding a functional MYO7A or CEP290 protein, wherein said nucleic acid molecule is operably linked to regulatory control elements that direct the transcription and translation thereof;

2) transducing photoreceptor cells with the CEP290-encoding vector and photoreceptor and retinal pigment epithelium cells with the MYO7A-encoding vector, whereby the expression of the MYO7A or CEP290 protein is induced in said cells.

In a further aspect, the invention relates to a recombinant adeno-associated viral (AAV) vector with AAV5 capsid, said vector carrying an expression cassette which contains a nucleic acid molecule encoding a functional MYO7A or CEP290 protein, wherein said nucleic acid molecule is operably linked to regulatory control elements that direct the transcription and translation thereof, for use in the treatment of retinal abnormalities and/or retinal dysfunction in a mammalian subject, preferably in a human individual affected by a disease associated with mutations in MYO7A or CEP290 genes.

In a preferred embodiment, said treatment comprises the transduction of photoreceptor cells with the CEP290-encoding vector and of photoreceptor and retinal pigment epithelium cells with the MYO7A-encoding vector, whereby the expression of the MYO7A or CEP290 protein is induced in said cells. In another preferred embodiment, the disease associated with mutations in MYO7A or CEP290 genes is selected from Usher Syndrome type IB and Leber congenital amaurosis.

Vectors with AAV5 capsids proved able to package genomes of up to 9 kb, preferably from about 4.7 to 9 kb, more efficiently than other serotypes, therefore their use for delivering the MYO7A or CEP290 genes according to the invention is preferred. The recombinant AAV2/5 vector, which is delivered to the subretinal space resulting in production of functional MYO7A or CEP290 proteins of the appropriate molecular weight and biological activity, is particularly preferred.

By “functional MYO7A or CEP290 proteins” applicant means that the MYO7A or CEP290 protein exhibits the function of the native protein, e.g.
the protein MYO7A correctly localizes to photoreceptors and retinal pigment epithelium and this results in correction of RPE melanosomes localization and rhodopsin localization in photoreceptors,
the protein CEP290 correctly localizes to photoreceptors and this expression results in inhibition of photoreceptor cell loss and increase of photoreceptor activity (as measured by electroretinograms—ERG).

Preferably, the functional MYO7A or CEP290 protein exhibits at least 50%, more preferably at least 80%, and most preferably at least 90% of the function of the native protein. Determination of the functional activities of MYO7A and CEP290 can be conducted, for example, in accordance with procedures described in Hashimoto T, et al. (“Lentiviral gene replacement therapy of retinas in a mouse model for Usher syndrome type 1B” Gene Ther. 2007 April; 14(7):584-94. Epub 2007 Feb. 1) and in Chang B, et al. (“In-frame deletion in a novel centrosomal/ciliary protein CEP290/NPHP6 perturbs its interaction with RPGR and results in early-onset retinal degeneration in the rd16 mouse” Hum Mol Genet. 2006 Jun. 1; 15(11):1847-57. Epub 2006 Apr. 21), hereby incorporated by reference, respectively.

For the purposes of this invention, a coding sequence of MYO7A or CEP290, which is preferably selected from SEQ ID NO:1 (MYO7A) and SEQ ID NO:2 (CEP290), or sequences encoding the same amino acid sequence due to the degeneracy of the genetic code, is functionally linked to a promoter sequence able to regulate the expression thereof in a mammalian retinal cell, particularly in photoreceptor cells. Suitable promoters that can be used according to the invention include the CMV (SEQ ID NO:3) and CBA (SEQ ID NO:4) promoters for controlling the transcription of the MYO7A sequence, the same promoters and additionally the human RHO (SEQ ID NO:5) promoter for controlling the transcription of the CEP290 sequence, fragments and variants thereof retaining a transcription promoter activity.

The construction of an AAV vector can be carried out following procedures and using techniques which are known to a person skilled in the art. The theory and practice for adeno-associated viral vector construction and use in therapy are illustrated in several scientific and patent publications (the following bibliography is herein incorporated by reference: Flotte T R. Adeno-associated virus-based gene therapy for inherited disorders. Pediatr Res. 2005 December; 58(6):1143-7; Goncalves M A. Adeno-associated virus: from defective virus to effective vector, Virol J. 2005 May 6; 2:43; Surace E M, Auricchio A. Adeno-associated viral vectors for retinal gene transfer. Prog Retin Eye Res. 2003 November; 22(6):705-19; Mandel R J, Manfredsson F P, Foust K D, Rising A, Reimsnider S, Nash K, Burger C. Recombinant adeno-associated viral vectors as therapeutic agents to treat neurological disorders. Mol Ther. 2006 March; 13(3):463-83).

In a further aspect, the invention relates to a pharmaceutical composition containing an AAV vector expressing the MYO7A or CEP290 coding sequence, preferably in a form suitable for ocular administration. Suitable administration forms include, but are not limited to, injectable solutions or suspensions, eye lotions and ophthalmic ointment. In a preferred embodiment, the AAV vector is administered by subretinal injection, e.g. by injection in the subretinal space, in the anterior chamber or in the retrobulbar space. Preferably the viral vectors are delivered via subretinal approach (as described in Bennicelli J, et al Mol Ther. 2008 Jan. 22; Reversal of Blindness in Animal Models of Leber Congenital Amaurosis Using Optimized AAV2-mediated Gene Transfer).

The doses of virus for use in therapy shall be determined on a case by case basis, depending on the administration route, the severity of the disease, the general conditions of the patients, and other clinical parameters. In general, suitable dosages will vary from 10⁹ to 10¹³ vg (vector genomes)/eye.

### DESCRIPTION OF THE FIGURES

FIG. 1. rAAV2/5 Packages Efficiently the MYO7A Gene

Average titers (genome copies/ml) of AAV serotypes containing the MYO7A cDNA. The AAV genome is composed of AAV2 ITRs, Cytomegalovirus (CMV) promoter and MYO7A cDNA sequence (rAAV genome size: 8.1 kb). Data are shown as average +/−standard error. The number of AAV preparations is four. The numbers on the top of the standard error bars represent the average titers.

FIG. 2. Genome Integrity of rAAV2/5-CMV-MYO7 and CEP290

Southern blot analysis of vector DNA isolated directly from rAAV large preps (2.5×10¹⁰ GC/lane) and separated on alkaline agarose gels. Lanes 1 and 2: genomes isolated from rAAV2/5-CMV-MYO7A; lanes 3 and 4: genomes isolated from rAAV2/5-CMV-CEP290. Samples in lanes 1 and 3 were treated with Dnase I.

FIG. 3. MYO7A Expression Following rAAV2/5 Delivery

Western blot analysis with anti-MYO7A antibodies of lysates from Cos7 cells. The cells were transduced with rAAV2/5-CMV-MYO7A (lane 1) or -EGFP (lane 2). Lysate from a control retina is loaded in lane 3. Molecular weights are indicated on the left. The amount of protein (micrograms, μg) loaded is indicated below each lane.

### EXPERIMENTAL SECTION

### Methods

Generation of the Plasmid Constructs

For the production of rAAV encoding MYO7A, the pAAV2.1-CMV-MYO7A was constructed as follows. Human MYO7A cDNA (6,648 bp, consisting of the MYO7A coding sequence without UTR regions) was cloned in the pAAV2.1-CMV-EGFP plasmid between the NotI and SacII sites (complete rAAV genome size: 8,107 bp). For this purpose, the MYO7A coding sequence (Genebank accession number: NM_{—}000260) was divided in three fragments of 2,853 bp, 2,275 bp and 1,890 bp, respectively, and amplified by PCR from cDNA of human retina (BD Biosciences) with the following oligos: F1 (NotI): ATTTGCGGCCGCATGGTGATTCTTCAGCAGGGG; R1: CCCCAGGAAGCCAAACATCT; F2: AGGGCTGAGTATCTGTGG; R2: CGGGGTTGGGGTTATCCT; F3: GCTGAGGACATTCGTGAC; R3 (SacII): TCCCCGCGGTCACTTGCCGCTCCTGGAG. Then, the three fragments, named F1, F2 and F3, were separately cloned in pZero Blunt Vector (Invitrogen), sequenced and then cloned via triple ligation reaction in pAAV2.1-CMV-EGFP. For the production of rAAV encoding CEP290, the pAAV2.1-CMV-CEP290 plasmid was produced as follows. The human CEP290 cDNA (7,440 bp, consisting of the CEP290 coding sequence, Genebank accession number: NM025114) was PCR amplified from a human osteosarcoma cell line (U2OS) cDNA and then cloned in the pAAV2.1-CMV-EGFP plasmid between the NotI and SacII sites (complete rAAV genome size: 8,900 bp).

rAAV Vector Production

Large preps of rAAV vectors were produced by the TIGEM AAV Vector Core using the pAAV2.1-CMV-EGFP, pAAV2.1-CMV-CEP290, pAAV-CMV-MYO7A. rAAV2/1, 2, 3, 4, 5, 7, 8 and 9 viruses were produced by triple transfection of 293 cells followed by two rounds of CsCl2 purification (24). For each viral preparation, physical titers [genome copies (GC)/ml] were determined by dot blot analysis (43) and by PCR quantification using TaqMan (Applied Biosystems, 42) by the TIGEM AAV Vector. For each large prep the titer was averaged from the dot blot and the TaqMan PCR quantification analyses.

Southern Blot Analyses of rAAV Vector DNA

DNA was extracted from 2.5×10¹⁰ viral particles (measured as genome copies). To digest unpackaged genomes, the vector solution was incubated with 11 μl of DNase (Roche) in a total volume of 250 containing 50 mM Tris pH7.5 and 1 mM MgCl₂ for 1 hr at 37° C. The DNase was then inactivated with 50 mM EDTA, followed by incubation at 50° C. for 45 min with proteinase K and 2.5% N-lauryl-sarcosyl solution to lyse the capsids. The DNA was extracted twice with phenol-chloroform and precipitated with 2 volumes of ethanol and 10% Sodium Acetate 3M. Alcaline agarose gel electrophoresis was performed as previously described (Sambrook, J. a. D. W. R. 2001. Molecular cloning: a laboratory manual. Cold Spring Harbor, N.Y.: Cold Spring Harbor Laboratory Press).

rAAV Infection of Cos7 Cells

Cos7 cells were infected with either rAAV2/5-CMV-MYO7A or rAAV2/5-CMV-EGFP (10⁴ GC/cell). Infected RPE cells were maintained in culture until MYO7A expression was assayed by Western blot at either 7 or 17 days post infection.

Analysis of MYO7A Expression by Western Blot.

Western blot was performed on Cos7 cells infectected with rAAV. Samples were lysed in SIE buffer [250 mM sucrose, 3 mM imidazoles (pH7.4), 1% ethanol and 1% NP-40] on ice for 30 min, proteins were denatured by heating at 37° C. for 30 min in sample buffer with 8M urea and separated by 6% SDS-PAGE. After blotting, specific proteins were labeled using anti-MYO7A antibodies.

Results

Vectors with AAV5 Capsids Efficiently Package MYO7A and CEP290 Constructs

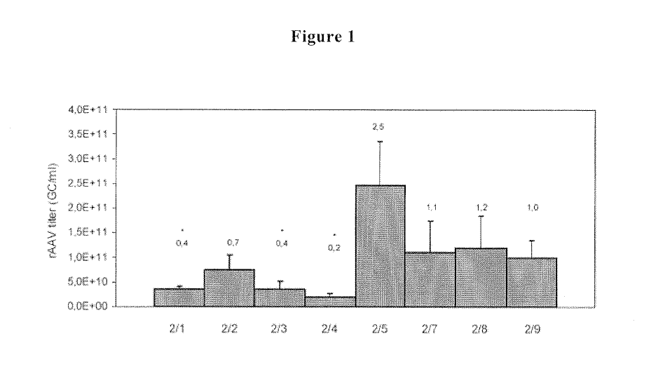

To test the ability of various AAV serotypes to package large genomes and to study whether the results obtained are dependent on the nucleotide composition of the sequence packaged, human MYO7A and CEP290 cDNA sequences were separately cloned between the AAV2 ITRs downstream of the Cytomegalovirus (CMV) promoter and upstream of a polyA signal. The resulting pAAV-CMV-MYO7A and -CEP290 constructs contained 8.1 and 8.9 kb respectively including the ITRs. The ability of various AAV serotypes to package the large genome containing the MYO7A gene was tested and rAAV2/5 vectors were found to be the most efficient (FIG. 1). rAAV2/5-CMV-MYO7A titers were 2.5×10¹¹±8.8×10¹⁰ GC/ml, n=4. Similarly, the titers of the rAAV2/5-CMV-CEP290 large preps were 5.1×10¹¹ GC/ml, n=2.

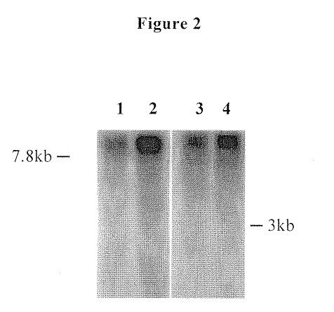

To demonstrate that the rAAV2/5-CMV-MYO7A and -CEP290 package their genome in its entire length, viral DNA was extracted from 2.5×10¹⁰ particles of each vector and analyzed by Southern blot following separation on alkaline agarose gel. DNAse-resistant bands of the expected molecular weight (8.9 kb for CEP290 and 8.1 kb for -MYO7A) were observed (FIG. 2).

Efficient In Vitro and In Vivo Transduction with rAAV2/5 Vectors Packaging Large Genes

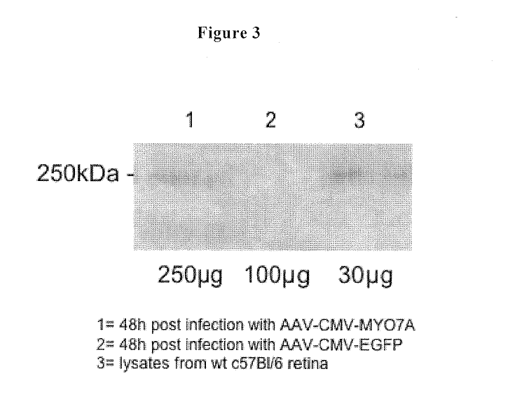

We evaluated MYO7A expression levels following AAV-mediated transduction of Cos7 cells. AAV2/5-CMV-MYO7A-mediated transduction results in efficient expression in vitro. Cos7 cells were transduced with either rAAV2/5-CMV-MYO7A or -EGFP (10⁴ GC/cell, FIG. 3). Western blot analysis with anti-MYO7A antibodies shows expression of the 250 kDa MYO7A band only in cells infected with rAAV2/5-CMV-MYO7A (similarly to a wild type retina loaded as positive control) but not with -EGFP.

1. A recombinant adeno-associated viral (AAV) vector with AAV5 capsid, carrying an expression cassette which contains a nucleic acid molecule encoding a functional MYO7A or CEP290 protein, wherein said nucleic acid molecule is functionally linked to a promoter sequence able to regulate its expression in mammalian retinal cells, for use in the treatment of retinal abnormalities and/or retinal dysfunction in a mammalian subject affected by a disease associated with mutations in MYO7A or CEP290 genes.

2. The recombinant vector according to claim 1, which is a AAV2/5 vector able to package up to 9 kb of nucleic acid.

3. The recombinant vector according to claim 1, wherein said nucleic acid molecule encoding MYO7A consists of SEQ ID NO:1, or a sequence encoding the same amino acid sequence as SEQ ID NO:1.

4. The recombinant vector according to claim 1, wherein said nucleic acid molecule encoding CEP290 consists of SEQ ID NO:2, or a sequence encoding the same amino acid sequence as SEQ ID NO:2.

5. The recombinant vector according to claim 1, wherein said promoter sequence is selected from SEQ ID NO:3, SEQ ID NO:4 and SEQ ID NO:5 fragments or variants thereof which retain a transcription promoter activity.

6. The recombinant vector according to claim 1, for use in the treatment of retinal abnormalities and/or retinal dysfunction in a mammalian subject affected by a disease associated with mutations in MYO7A or CEP290 genes, wherein said treatment comprises the transduction of photoreceptor cells with the CEP290-encoding vector and of photoreceptor and retinal pigment epithelium cells with the MYO7A-encoding vector, whereby the expression of the MYO7A or CEP290 protein is induced in said cells.

7. The recombinant vector according to claim 1, for use in the treatment of retinal abnormalities and/or retinal dysfunction in a human subject affected by a disease associated with mutations in MYO7A or CEP290 genes, wherein said disease is selected from Usher Syndrome type IB and Leber congenital amaurosis.

8. A pharmaceutical preparation containing an AAV vector as defined in claim 1, in a form suitable for ocular administration.

9. The pharmaceutical composition according to claim 8, which is in the form of injectable solution.

10. A method for correcting retinal abnormalities and/or retinal function in a mammalian subject affected by a disease associated with mutations in MYO7A or CEP290 genes, said method comprising the steps of:
1) providing a recombinant adeno-associated viral (AAV) vector with AAV5 capsid, said vector carrying an expression cassette which contains a nucleic acid molecule encoding a functional MYO7A or CEP290 protein, wherein said nucleic acid molecule is operably linked to regulatory control elements that direct the transcription and translation thereof;
2) transducing photoreceptor cells with the CEP290-encoding vector, photoreceptor and retinal pigment epithelium cells with the MYO7A-encoding vector, whereby the expression of the MYO7A or CEP290 protein is induced in said cells.

11. The method according to claim 10, wherein said subject is human.

12. The method according to claim 10, wherein said disease is selected from Usher Syndrome type IB and Leber congenital amaurosis.

13. The method according to claim 10, wherein said vector with AAV5 capsid is able to package up to 9 kb of nucleic acid.

14. The method according to claim 13, wherein said vector is AAV2/5.

15. The method according to claim 10, wherein said recombinant adeno-associated viral (AAV) vector with AAV5 capsid carries an expression cassette in which a coding sequence of MYO7A or CEP290 is functionally linked to a promoter sequence able to regulate its expression in mammalian retinal cells.

16. The method according to claim 15, wherein said coding sequence of MYO7A consists of SEQ ID NO:1, or a sequence encoding the same amino acid sequence as SEQ ID NO:1.

17. The method according to claim 15, wherein said coding sequence of CEP290 consists of SEQ ID NO:2, or a sequence encoding the same amino acid sequence as SEQ ID NO:2.

18. The method according to claim 15, wherein said promoter sequence is selected from SEQ ID NO:3, SEQ ID NO:4 and SEQ ID NO:5 fragments or variants thereof which retain a transcription promoter activity.

19. The method according to claim 1, wherein transduction of retinal pigment epithelium and photoreceptor cells is effected by subretinal administration of said vector or a pharmaceutical preparation thereof.


SEQUENCE LISTING (11 sequences)

SEQ ID NO: 1 - DNA, 6648 bp, Homo sapiens
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccacctcgtgagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaatgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 2 - DNA, 7440 bp, Homo sapiens
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 3 - DNA, 776 bp, Cytomegalovirus sequence
gtagccatgctctggaagatcttcaatatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactaga

SEQ ID NO: 4 - DNA, 672 bp, Unknown
  Description of Unknown CBA promoter sequence
gtgccacctggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 5 - DNA, 805 bp, Homo sapiens
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcc

SEQ ID NO: 6 - DNA, 33 bp, Artificial Sequence
  Description of Artificial Sequence Synthetic oligonucleotide
atttgcggccgcatggtgattcttcagcagggg

SEQ ID NO: 7 - DNA, 20 bp, Artificial Sequence
  Description of Artificial Sequence Synthetic oligonucleotide
ccccaggaagccaaacatct

SEQ ID NO: 8 - DNA, 18 bp, Artificial Sequence
  Description of Artificial Sequence Synthetic oligonucleotide
agggctgagtatctgtgg

SEQ ID NO: 9 - DNA, 18 bp, Artificial Sequence
  Description of Artificial Sequence Synthetic oligonucleotide
cggggttggggttatcct

SEQ ID NO: 10 - DNA, 18 bp, Artificial Sequence
  Description of Artificial Sequence Synthetic oligonucleotide
gctgaggacattcgtgac

SEQ ID NO: 11 - DNA, 28 bp, Artificial Sequence
  Description of Artificial Sequence Synthetic oligonucleotide
tccccgcggtcacttgccgctcctggag